(19) United States
(12) Patent Application Publication
(10) Pub. No.: US 2010/0178342 A1
(43) Pub. Date: Jul. 15, 2010

(54) Solid Pharmaceutical Preparation

(75) Inventors: Ulrich Brauns, Biberach (DE); Thomas Friedl, Ochsenhausen (DE); Sabine Landerer, Biberach (DE)

Correspondence Address: MICHAEL P. MORRIS;BOEHRINGER INGELHEIM USA CORPORATION, 900 RIDGEBURY ROAD, RIDGEFIELD, CT, 06877-0368, US

(73) Assignee: BOEHRINGER INGELHEIM INTERNATIONAL GMBH, Ingelheim (DE)

(21) Appl. No.: 12/730,831

(22) Filed: Mar. 24, 2010

Related U.S. Application Data
(63) Continuation of application No. 10/987,831, filed on Nov. 12, 2004.

(30) Foreign Application Priority Data
Nov. 18, 2003 (DE) 1035 832
Mar. 11, 2004 (DE) 102004012045

(51) Int. Cl. A61K 9/28; A61K 31/46; A61P 25/16; A61P 25/28; B05D 1/02

(52) U.S. Cl. 424/474; 514/304; 427/2.14

(57) ABSTRACT

The invention relates to a solid pharmaceutical preparation containing one or more solid carriers and/or excipients and an active substance selected from among the Monoamine Neurotransmitter Re-uptake Inhibitors which have a 2,3-disubstituted tropane structure, the preparation thereof and use thereof for preparing a pharmaceutical composition for the treatment or prevention of central-nervous diseases or disorders.

### CROSS-REFERENCE TO RELATED APPLICATIONS

The present application is a continuation of U.S. application Ser. No. 10/987,831, filed Nov. 12, 2004, the entirety of which is incorporated by reference herein.

### TECHNICAL FIELD

The invention relates to a solid pharmaceutical preparation containing one or more solid carriers and/or excipients and an active substance from the group of the Monoamine Neurotransmitter Re-uptake Inhibitors with a 2,3-disubstituted tropane structure, the preparation thereof and use thereof for preparing a pharmaceutical composition for the treatment or prevention of central-nervous diseases or disorders.

### PRIOR ART

Monoamine Neurotransmitter Re-uptake Inhibitors, which have a 2,3-disubstituted tropane structure, are compounds with pharmacologically valuable properties. They may provide great therapeutic benefit for example in the treatment of central-nervous problems such as dementia connected with Alzheimer's disease or Parkinson's disease.

Such compounds are known e.g. from International Patent Applications WO 93/09814 and WO 97/30997, in which different formulations for such compounds are also proposed.

In view of the very high activity potential of these compounds, there is a need for formulations with high stability and a low content of active substance. Because of the small amount of active substance, such formulations make high demands of the manufacturing process in terms of uniformity of content. The high uniformity of content needed cannot easily be achieved with conventional production processes such as direct tabletting or wet granulation.

The objective on which the present invention is based is thus to provide a solid pharmaceutical formulation for Monoamine Neurotransmitter Re-uptake Inhibitors which have a 2,3-disubstituted tropane structure, with high stability, rapid dissolving in-vitro and good bioavailability as well as high uniformity of content.

It has now surprisingly been found that the disadvantages of formulations produced in the conventional manner, particularly with regard to the uniformity of content, can be overcome if a solution of an active substance selected from among the Monoamine Neurotransmitter Re-uptake Inhibitors which have a 2,3-disubstituted tropane structure is sprayed onto a carrier and/or the formulation or the spray medium contains a moisture binder.

### BRIEF SUMMARY OF THE INVENTION

The invention thus relates to a solid pharmaceutical preparation containing one or more solid carriers and/or excipients and an active substance selected from among the Monoamine Neurotransmitter Re-uptake Inhibitors with a 2,3-disubstituted tropane structure, which

(a) may be obtained by spraying a solution of the active substance onto at least one carrier; and
(b) optionally contains one or more moisture binders, preferably in the spray solution.

The invention further relates to a process for preparing pharmaceutical preparations of this kind, by

(a) dissolving an active substance selected from among the Monoamine Neurotransmitter Re-uptake Inhibitors, which has a 2,3-disubstituted tropane structure, in a suitable solvent optionally in the presence of an excipient;
(b) spraying the resulting solution onto one or more solid carriers;
(c) optionally adding other carriers and excipients;
(d) shaping and optionally compressing the resultant mixture; and
(e) optionally applying a suitable film coating.

Finally, the invention relates to the use of a pharmaceutical preparation according to one of claims 1 to for preparing a pharmaceutical composition for the treatment or prevention of central-nervous diseases or disorders selected from the group consisting of depression, all types of dementia, Parkinson's disease or obesity.

### BRIEF DESCRIPTION OF THE FIGURES

FIG. 1 illustrates the dissolving characteristics of a pharmaceutical preparation according to the invention in the form of a film-coated tablet with and without moisture binders containing 1 mg of a compound of formula IA at a pH of 1.2.

FIG. 2 illustrates the dissolving characteristics of a pharmaceutical preparation according to the invention in the form of a film-coated tablet with and without moisture binders containing 1 mg of a compound of formula IA at a pH of 6.8.

### DETAILED DESCRIPTION OF THE INVENTION

As a rule, Monoamine Neurotransmitter Re-uptake Inhibitors with a 2,3-disubstituted tropane structure are those of formula (I), as disclosed for example in International Patent Applications WO 93/09814, as well as WO 97/30997, which is equivalent U.S. Pat. No. 6,288,079, all of which are incorporated herein by reference in their entirties:

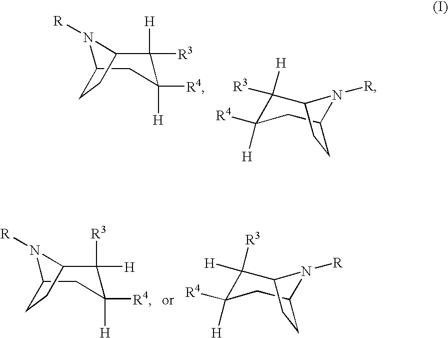

or a tautomer, a pharmaceutically acceptable salt, solvate, or a physiological functional derivative thereof, wherein

R is hydrogen, alkyl, alkenyl, alkynyl, cycloalkyl, cycloalkylalkyl or 2-hydroxyethyl;

R³ is CH₂—X—R′,
where X denotes O, S, or NR″; wherein
   R″ is hydrogen or alkyl; and
R′ is alkyl, alkenyl, alkynyl, cycloalkyl, cycloalkylalkyl, or —CO-alkyl; heteroaryl, which may be mono- or polysubstituted by alkyl, cycloalkyl, or cycloalkylalkyl;
   phenyl, which may be mono- or polysubstituted by a substituent selected from among halogen, CF₃, CN, alkoxy, alkyl, alkenyl, alkynyl, amino, nitro, and heteroaryl;
   phenylphenyl;
   pyridyl, which may be mono- or polysubstituted by a substituent selected from among halogen, CF₃, CN, alkoxy, alkyl, alkenyl, alkynyl, amino, nitro, and heteroaryl;
   thienyl, which may be mono- or polysubstituted by a substituent selected from among halogen, CF₃, CN, alkoxy, alkyl, alkenyl, alkynyl, amino, nitro, and heteroaryl; or
   benzyl, which may be mono- or polysubstituted by a substituent selected from among halogen, CF₃, CN, alkoxy, alkyl, alkenyl, alkynyl, amino, nitro, and heteroaryl; or
   (CH₂)ₙCO₂R¹¹, COR¹¹, or CH₂R¹², wherein
      R¹¹ is alkyl, cycloalkyl, or cycloalkylalkyl;
         phenyl, which may be mono- or polysubstituted by a substituent selected from among halogen, CF₃, CN, alkoxy, alkyl, alkenyl, alkynyl, amino, nitro, and
         heteroaryl;
         phenylphenyl;
         pyridyl, which may be mono- or polysubstituted by a substituent selected from among: halogen, CF₃, CN, alkoxy, alkyl, alkenyl, alkynyl, amino, nitro, and heteroaryl; or
         thienyl which may be mono- or polysubstituted by a substituent selected from among halogen, CF₃, CN, alkoxy, alkyl, alkenyl, alkynyl, amino, nitro, and heteroaryl; or
         benzyl;
      n is 0 or 1; and
      R¹² is O-phenyl, which may be mono- or polysubstituted by a substituent selected from among halogen, CF₃, CN, alkoxy, alkyl, alkenyl, alkynyl, amino, nitro, and heteroaryl; or
         O—CO-phenyl, which may be mono- or polysubstituted by a substituent selected from among halogen, CF₃, CN, alkoxy, alkyl, alkenyl, alkynyl, amino, nitro, and heteroaryl; or
         CH═NOR′; wherein
         R′ is hydrogen, alkyl, cycloalkyl, cycloalkylalkyl, alkenyl, alkynyl, or aryl, which may in turn be substituted by —COON, —COO-alkyl, —COO-cycloalkyl, or phenyl, which may be mono- or polysubstituted by a substituent selected from among halogen, CF₃, CN, alkoxy, alkyl, alkenyl, alkynyl, amino, and nitro;

R⁴ is phenyl, 3,4-methylenedioxyphenyl, benzyl, naphthyl, or heteroaryl, which may be mono- or polysubstituted by a substituent selected from among halogen, CF₃, CN, alkoxy, alkyl, alkenyl, alkynyl, amino, nitro, and heteroaryl.

Preferred are compounds of formula I wherein:
R³ is 1,2,4-oxadiazol-3-yl, which may be substituted in the 5 position by
   alkyl, cycloalkyl, or cycloalkylalkyl;
   phenyl, which may be mono- or polysubstituted by a substituent selected from among halogen, CF₃, CN, alkoxy, alkyl, alkenyl, alkynyl, amino, nitro, and heteroaryl;
   phenylphenyl; or
   benzyl, which may be mono- or polysubstituted by a substituent selected from among halogen, CF₃, CN, alkoxy, alkyl, alkenyl, alkynyl, amino, nitro, and heteroaryl; or
R³ is 1,2,4-oxadiazol-5-yl, which may be substituted in the 3 position by
   alkyl, cycloalkyl, or cycloalkylalkyl;
   phenyl, which may be mono- or polysubstituted by a substituent selected from among halogen, CF₃, CN, alkoxy, alkyl, alkenyl, alkynyl, amino, nitro, and heteroaryl;
   phenylphenyl; or
   benzyl, which may be mono- or polysubstituted by a substituent selected from among halogen, CF₃, CN, alkoxy, alkyl, alkenyl, alkynyl, amino, nitro, and heteroaryl.

In another preferred embodiment of the compounds of general formula I R³ is CH₂—X—R′, wherein

X is O, S, or NR″; while R″ denotes hydrogen or alkyl; and

R′ denotes alkyl, alkenyl, alkynyl, cycloalkyl, cycloalkylalkyl, or —CO-alkyl.

Also preferred are the compounds of formula (I), wherein
R³ is CH═NOR′; where
R′ denotes hydrogen; alkyl, cycloalkyl, cycloalkylalkyl, alkenyl, alkynyl or aryl, which may be substituted by a substituent selected from among: —COON, —COO-alkyl, —COO-cycloalkyl, and phenyl, which may be mono- or polysubstituted by a substituent selected from among halogen, CF₃, CN, alkoxy, alkyl, alkenyl, alkynyl, amino, and nitro.

Also preferred are the compounds of formula (I), wherein
R⁴ denotes phenyl which may be mono- or disubstituted by a substituent selected from among halogen, CF₃, CN, alkoxy, alkyl, alkenyl, alkynyl, amino, nitro, and heteroaryl.

Particularly preferred are the compounds of formula (I), wherein R⁴ denotes phenyl, which is mono- or disubstituted by chlorine.

Also preferred are those 2,3-disubstituted tropane derivatives with a Monoamine Neurotransmitter Re-uptake inhibiting activity which have a (1R,2R,3S) configuration.

Particularly preferred are the compounds of formula (I), wherein R³ is
—CH₂—X—R′, where X is O or S, and R′ denotes methyl, ethyl, propyl, or cyclopropylmethyl;
—CH═NOR′; where R′ denotes hydrogen or alkyl; or
1,2,4-oxadiazol-5-yl, which may be substituted by alkyl in the 3 position.

Preferably, also, R denotes hydrogen, methyl, ethyl, or propyl.

Preferred compounds of formula I are those wherein R⁴ is 3,4-dichlorophenyl.

Also preferred are the compounds of formula I1,

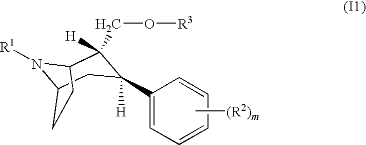

wherein
R¹ denotes a hydrogen atom or a C₁₋₆ alkyl group, particularly hydrogen, methyl, or ethyl;
R² denotes a halogen atom or a CF₃ or cyano group, particularly fluorine, chlorine, or bromine;
R³ denotes a hydrogen atom, or a C₁₋₆ alkyl group, or C₃₋₆-cycloalkyl-C₁₋₃-alkyl group, particularly methyl, ethyl, or propyl; and
m is 0 or an integer from 1 to 3, particularly 1 or 2;
   or a tautomer, a pharmaceutically acceptable salt, solvate, or a physiological functional derivative thereof.

The term “C₁₋₆ alkyl” as used above and hereinafter comprises methyl and ethyl groups, as well as straight-chain and branched propyl, butyl, pentyl, and hexyl groups. Particularly preferred alkyl groups are methyl, ethyl, n-propyl, isopropyl, and t-butyl.

The term “C₃₋₆ cycloalkyl” as used above and hereinafter comprises cyclic propyl, butyl, pentyl, and hexyl groups such as cyclopropyl and cyclohexyl.

The term “halogen” as used above and hereinafter includes fluorine, chlorine, bromine, and iodine, of which fluorine and chlorine are particularly preferred.

The term “physiologically functional derivative” as used above and hereinafter encompasses derivatives which are obtained from the compounds of formula (I) under physiological conditions, such as, for example, N-oxides.

The term “pharmaceutically acceptable acid addition salts” as used above and hereinafter encompasses acid addition salts that are formed with hydrochloric acid, bromic acid, sulphuric acid, phosphoric acid, methanesulphonic acid, acetic acid, fumaric acid, succinic acid, lactic acid, citric acid, tartaric acid, and maleic acid; the salts of hydrochloric acid, bromic acid, sulphuric acid, phosphoric acid, acetic acid, and citric acid are particularly preferred. Most preferred is the salt of citric acid.

In a particularly preferred embodiment the compounds of formula (I) are selected from the group comprising:
(1R,2R,3S)-2-(3-cyclopropyl-1,2,4-oxadiazol-5-yl)-3-(4-fluorophenyl)tropane;
(1R,2R,3S)-2-(3-phenyl-1,2,4-oxadiazol-5-yl)-3-(4-fluorophenyl)tropane;
(1R,2R,3S)-2-(3-phenyl-1,2,4-oxadiazol-5-yl)-3-(4-methylphenyl)-tropane;
(1R,2R,3S)-2-(3-benzyl-1,2,4-oxadiazol-5-yl)-3-(4-fluorophenyl)tropane;
(1R,2R,3S)-2-(3-(4-phenyl-phenyl)-1,2,4-oxadiazol-5-yl)-3-(4-fluorophenyl)tropane;
(1R,2R,3S)-2-(3-phenyl-1,2,4-oxadiazol-5-yl)-3-(2-naphthyl)tropane;
(1R,2R,3S)-3-(3,4-dichlorophenyl)tropane-2-aldoxime;
(1R,2R,3S)-3-(3,4-dichlorophenyl)-tropane-2-O-methyl-aldoxime;
(1R,2R,3S)-3-(3,4-dichlorophenyl)tropane-2-O-benzyl-aldoxime;
(1R,2R,3S)-3-(3,4-dichlorophenyl)tropane-2-O-ethoxycarbonylmethyl-aldoxime;
(1R,2R,3S)-3-(3,4-dichlorophenyl)tropane-2-O-methoxycarbonylmethyl-aldoxime;
(1R,2R,3S)-3-(3,4-dichlorophenyl)tropane-2-O-(1-ethoxycarbonyl-1,1-dimethyl-methyl)-aldoxime;
(1R,2R,3S)-3-(3,4-dichlorophenyl)tropane-2-O-carboxymethyl-2-aldoxime;
(1R,2R,3S)—N-normethyl-3-(3,4-dichlorophenyl)tropane-2-O-methyl-aldoxime;
(1R,2R,3S)—N-normethyl-3-(3,4-dichlorophenyl)tropane-2-O-benzyl-aldoxime;
(1R,2R,3S)-3-(4-methylphenyl)tropane-2-O-methyl-aldoxime;
(1R,2R,3S)-3-(3,4-dichlorophenyl)tropane-2-O-(1,1-dimethylethyl)-aldoxime;
(1R,2R,3S)-3-(4-chlorophenyl)tropane-2-O-aldoxime;
(1R,2R,3S)-3-(4-chlorophenyl)tropane-2-O-methylaldoxime hydrochloride;
(1R,2R,3S)-3-(4-chlorophenyl)tropane-2-O-methoxycarbonylmethyl-aldoxime;
(1R,2R,3S)-3-(3,4-dichlorophenyl)tropane-2-O— (2-propynyl)-aldoxime;
(1R,2R,3S)-3-(3,4-dichlorophenyl)tropane-2-O-(2-methylpropyl)-aldoxime;
(1R,2R,3S)-3-(3,4-dichlorophenyl)tropane-2-β-cyclopropylmethyl-aldoxime;
(1R,2R,3S)-3-(3,4-dichlorophenyl)tropane-2-O-ethyl-aldoxime;
(1R,2R,3S)-2-methoxymethyl-3-(3,4-dichlorophenyl)-tropane;
(1R,2R,3S)-2-isopropoxymethyl-3-(3,4-dichlorophenyl)-tropane;
(1R,2R,3S)-2-ethoxymethyl-3-(3,4-dichlorophenyl)-tropane;
(1R,2R,3S)-2-ethoxymethyl-3-(3,4-dichlorophenyl)-nortropane;
(1R,2R,3S)-2-cyclopropylmethyloxymethyl-3-(3,4-dichlorophenyl)-tropane;
(1R,2R,3S)-2-methoxymethyl-3-(4-chlorophenyl)-tropane;
(1R,2R,3S)—N-normethyl-2-methoxymethyl-3-(4-chlorophenyl)-tropane;
(1R,2R,3S)-2-ethoxymethyl-3-(4-chlorophenyl)-tropane;
(1R,2R,3S)—N-normethyl-2-methoxymethyl-3-(3,4-dichlorophenyl)-tropane;
(1R,2R,3S)—N-normethyl-2-ethoxymethyl-3-(3,4-dichlorophenyl)-tropane;
(1R,2R,3S)—N-normethyl-2-ethoxymethyl-3-(4-chlorophenyl)-tropane;
(1R,2R,3S)—N-normethyl-2-cyclopropylmethyloxymethyl-3-(4-chlorophenyl)-tropane;
(1R,2R,3S)-2-cyclopropylmethyloxymethyl-3-(4-chlorophenyl)-tropane;
(1R,2R,3S)-2-ethylthiomethyl-3-(3,4-dichlorophenyl)-tropane;
(1R,2R,3S)-2-hydroxymethyl-3-(4-fluorophenyl)tropane;
(1R,2R,3S)-2-hydroxymethyl-3-(3,4-dichlorophenyl)tropane;
(1R,2R,3S)—N-normethyl-N-(tert-butoxycarbonyl)-2-hydroxymethyl-3-(3,4-dichlorophenyl)tropane;
(1R,2R,3S)-2-hydroxymethyl-3-(4-chlorophenyl)tropane;
(1R,2R,3S)-2-(3-(2-furanyl)-1,2,4-oxadiazol-5-yl)-3-(3,4-dichlorophenyl)-tropane;
(1R,2R,3S)-2-(3-(3-pyridyl)-1,2,4-oxadiazol-5-yl)-3-(3,4-dichlorophenyl)-tropane;
(1R,2R,3S)—N-normethyl-N-allyl-2-(3-(4-pyridyl)-1,2,4-oxadiazol-5-yl)-3-(3,4-dichlorophenyl)-tropane;
(1R,2R,3S)—N-normethyl-N-ethyl-2-(3-(4-pyridyl)-1,2,4-oxadiazol-5-yl)-3-(3,4-dichlorophenyl)-tropane;
(1R,2R,3S)—N-normethyl-N-(2-hydroxyethyl)-2-(3-(4-pyridyl)-1,2,4-oxadiazol-5-yl)-3-(3,4-dichlorophenyl)-tropane;
(1R,2R,3S)—N-normethyl-2-(3-(4-pyridyl)-1,2,4-oxadiazol-5-yl)-3-(3,4-dichlorophenyl)-tropane;
(1R,2R,3S)—N-normethyl-N-allyl-2-(3-(3-pyridyl)-1,2,4-oxadiazol-5-yl)-3-(3,4-dichlorophenyl)-tropane;
(1R,2R,3S)—N-normethyl-N-allyl-2-(3-(2-pyridyl)-1,2,4-oxadiazol-5-yl)-3-(3,4-dichlorophenyl)-tropane;
(1R,2R,3S)-2-(3-(2-thienyl)-1,2,4-oxadiazol-5-yl)-3-(4-chlorophenyl)-tropane;
(1R,2R,3S)-2-(3-(2-thienyl)-1,2,4-oxadiazol-5-yl)-3-(3,4-dichlorophenyl)-tropane;
(1R,2R,3S)-2-(3-(4-pyridyl)-1,2,4-oxadiazol-5-yl)-3-(3,4-dichlorophenyl)-tropane;
(1R,2R,3S)-2-(3-(2-pyridyl)-1,2,4-oxadiazol-5-yl)-3-(3,4-dichlorophenyl)-tropane;
(1R,2R,3S)-2-(3-(4-pyridyl)-1,2,4-oxadiazol-5-yl)-3-(4-chlorophenyl)-tropane;
(1R,2R,3S)-2-(3-(3-pyridyl)-1,2,4-oxadiazol-5-yl)-3-(4-chlorophenyl)-tropane;
(1R,2R,3S)-2-(3-2-pyridyl)-1,2,4-oxadiazol-5-yl)-3-(4-chlorophenyl)-tropane;
(1R,2R,3S)-2-(3-phenyl-1,2,4-oxadiazol-5-yl)-3-(4-fluorophenyl)-tropane;
(1R,2R,3S)-2-(3-phenyl-1,2,4-oxadiazol-5-yl)-3-(4-methylphenyl)-tropane;
(1R,2R,3S)-2-(3-benzyl-1,2,4-oxadiazol-5-yl)-3-(4-fluorophenyl)-tropane;
(1R,2R,3S)-2-(3-(4-phenylphenyl)-1,2,4-oxadiazol-5-yl)-3-(4-fluorophenyl)-tropane;
(1R,2R,3S)-2-(3-phenyl-1,2,4-oxadiazol-5-yl)-3-(2-naphthyl)-tropane;
(1R,2R,3S)-2-(4-chlorophenoxy-methyl)-3-(4-fluorophenyl)-tropane;
(1R,2R,3S)-2-(4-chlorophenoxy-methyl)-3-(4-fluorophenyl)-tropane;
(1R,2R,3S)-2-(4-chlorophenoxy-methyl)-3-(3,4-dichlorophenyl)-tropane;
(1R,2R,3S)-2-(4-chlorophenoxy-methyl)-3-(4-methylphenyl)-tropane;
(1R,2R,3S)-2-(4-benzoyloxy-methyl)-3-(4-fluorophenyl)-tropane;
(1R,2R,3S)-2-carbomethoxy-3-(2-naphthyl)-tropane;
(1R,2R,3S)-2-carbomethoxy-3-(3,4-dichlorophenyl)-tropane;
(1R,2R,3S)-2-carbomethoxy-3-benzyl-tropane;
(1R,2R,3S)-2-carbomethoxy-3-(4-chlorophenyl)-tropane;
(1R,2R,3S)-2-carbomethoxy-3-(4-methylphenyl)-tropane;
(1R,2R,3S)-2-carbomethoxy-3-(1-naphthyl)-tropane;
(1R,2R,3S)-2-carbomethoxy-3-(4-phenylphenyl)-tropane;
(1R,2R,3S)-2-carbomethoxy-3-(4-t-butyl-phenyl)-tropane;
(1R,2R,3S)-2-(4-fluorobenzoyl)-3-(4-fluorophenyl)-tropane;
   or a tautomer, a pharmaceutically acceptable salt, solvate, or a physiological functional derivative thereof.

Most preferred is the compound of formula IA

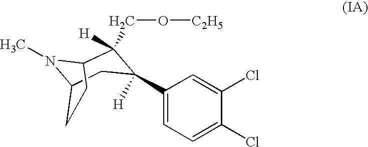

or a tautomer, a pharmaceutically acceptable salt, solvate, or a physiological functional derivative thereof, particularly the citrate thereof.

Preferably the pharmaceutical preparations according to the invention contains up to 5.00 wt. %, preferably 0.01 to 3.00 wt. %, particularly 0.00 to 1.50 wt. %, most preferably 0.10 to 0.80 wt. % of an active substance selected from among the Monoamine Neurotransmitter Re-uptake Inhibitors with a 2,3-disubstituted tropane structure, the percentages referring to the particular salt of the active substance used.

Also preferred is a pharmaceutical preparation form which may be obtained by spraying a solution of the active substance, while the solvent contains water, an alcohol and optionally a moisture binder. The ratio of the solvents alcohol and water may be from 100:0 to 0:100 (wt. %), preferably 20:80 to 80:20 (wt. %), particularly preferably 40:60 to 60:40 (wt. %).

Preferred moisture binders are polyvinylpyrrolidone (Povidone), copolymers of vinylpyrrolidone with other vinyl derivatives (Copovidone), cellulose derivatives such as methylhydroxypropylcellulose, methylcellulose, or hydroxypropylcellulose, particularly hydroxypropylcellulose (HPC).

In another preferred embodiment the active substance is precipitated in predominantly crystalline form on the carrier material when sprayed.

Within the scope of the present invention, carbohydrates such as lactose or mannose, particularly finely divided lactose and lactose monohydrate, but also sugar alcohols, such as mannitol, sorbitol, or xylitol, particularly mannitol, are of particular importance as carrier materials. These carriers have proved particularly advantageous in the formulation according to the invention. In a preferred aspect, therefore, the present invention relates to a preparation form containing at least one compound of formula I, that contains, beside the active substance lactose, in particular, finely divided lactose and lactose monohydrate as carrier material.

According to the invention, the weight ratio between the component lactose contained in the tablet to the active substance is in the range from about 200:1 to about 20:1. Preferably, the ratio of lactose to the active substance is in the range from about 150:1 to about 50:1. Preferably, the proportion by weight of lactose based on the total mass of the tablet according to the invention is in the range from about 50-80 wt. %, preferably between about 55-75 wt. %.

Also preferred are pharmaceutical preparation forms, wherein the carrier materials are selected from among the carbohydrates and dry binders.

The term “dry binder” above and hereinafter denotes excipients that are suitable for binding other components to one another. Preferred binders according to the invention are selected from the group comprising:

powdered cellulose, microcrystalline cellulose, sorbitol, starch, polyvinylpyrrolidone (Povidone), copolymers of vinylpyrrolidone with other vinyl derivatives (Copovidone), cellulose derivatives, particularly methylhydroxypropylcellulose, e.g. Methocel E 5 P, and mixtures of these compounds. Preferably, powdered cellulose, particularly microcrystalline cellulose and/or Copovidone, are present as binders. Most preferred is microcrystalline cellulose.

Thanks to this particularly preferred carrier combination of microcrystalline cellulose, anhydrous lactose, and lactose monohydrate, tablets are obtained having good mechanical stability and at the same time rapid release of active substance and good bioavailability.

If one of the above-mentioned dry binders is added to the formulation according to the invention, the weight ratio of lactose to binder is preferably about 5:1 to about 1:2, preferably about 3:1 to about 1:1, particularly preferably about 2.5:1 to 1.5:1.

Also preferred are pharmaceutical preparation forms in which the excipients are selected from the group consisting of moisture binders, lubricants, breakdown agents, parting compounds, and wetting agents.

Within the scope of the present invention, these breakdown agents may also be referred to as disintegrants. These are preferably selected according to the invention from the group comprising: sodium starch glycolate, cross-linked polyvinylpyrrolidone (Crospovidone), croscarmellose sodium salt (cellulose carboxymethylether sodium salt, cross-linked), carboxymethylcellulose, dried maize starch, and mixtures thereof. Within the scope of the present invention, it is particularly preferable to use sodium starch glycolate, Crospovidone, and, preferably, croscarmellose sodium salt. If the above-mentioned breakdown agents are used, the amount thereof by weight, based on the total mass of the tablet according to the invention, is preferably in the range from about 0.5-10 wt. %, most preferably about 1.0-5.0 wt. %.

Lubricants that may be used within the scope of the present invention include, for example, silicon dioxide, talc, stearic acid, sodium stearylfumarate, magnesium stearate, and glycerol tribehenate. Preferably, according to the invention, vegetable magnesium stearate is used. If the above-mentioned flow or flow regulating agents or lubricants are used, the amount thereof by weight, based on the total mass of the formulation according to the invention, is preferably in the range from about 0.1-10 wt. %, preferably about 0.5-5 wt. %, particularly preferably between 0.6 and 1.0 wt. %.

In a preferred embodiment, the preparation form according to the invention is a tablet, particularly a film-coated tablet.

As a rule, the film coating essentially consists of one or more film-forming agents, one or more agents for increasing elasticity, so-called plasticizers, one or more parting compounds, one or more pigments, and, optionally, one or more colorings.

Preferred film-coated tablets are those wherein the film coating consists essentially of

35 to 65 wt. % of at least one film-forming agent, particularly HPMC;
3.5 to 10% wt. % of at least one agent for increasing elasticity, particularly PEG;
5 to 20 wt. % of at least one coating, particularly a silicate;
10 to 40 wt. % of at least one pigment, particularly titanium dioxide
0 to 10% wt. % of at least one coloring, particularly iron oxides, based on the total mass of the film coating.

A preferred pharmaceutical preparation form according to one of the preceding claims is characterised in that it consists essentially of the following components:

an active substance selected from among the Monoamine Neurotransmitter Re-uptake Inhibitors which have a 2,3-disubstituted tropane structure, preferably a compound of formula (I), particularly the compound of formula (IA);

one or more carrier materials selected from the group consisting of carbohydrates and dry binders, preferably lactose and cellulose;

one or more excipients selected from the group consisting of cellulose derivatives and salts of fatty acids, preferably HMC, CMC Na, cross-linked, and magnesium stearate;

a film coating which consists essentially of one or more film-forming agents, one or more agents for increasing elasticity, one or more parting compounds, one or more pigments and optionally one or more colourings.

Particularly preferred is a pharmaceutical preparation in the form of a film-coated tablet, which consists essentially of the following components:

0.01 to 5.00 wt. % of an active substance selected from among the Monoamine Neurotransmitter Re-uptake Inhibitors which have a 2,3-disubstituted tropane structure, particularly 0.02 to 3.00 wt. % of an active substance of formula I;

80.00 to 95.00 wt. % of one or more carrier materials selected from the group consisting of carbohydrates and dry binders, particularly carrier materials consisting of:

27.5 to 32.5 wt. % anhydrous lactose;

27.5 to 32.5 wt. % lactose monohydrate;

25.0 to 30.0 wt. % microcrystalline cellulose;

1.00 to 10.00 wt. % of one or more excipients selected from the group consisting of cellulose derivatives and salts of fatty acids, particularly 2.00 to 8.00 wt. % of one or more excipients selected from the group consisting of HPC, CMC Na, cross-linked, and magnesium stearate;

0 to 10.00 wt. % of a film coating consisting essentially of one or more film-forming agents, one or more plasticisers, 1.00 to 5.00 wt. % of a film coating comprising HPMC, MHPC, PEG, one or more silicates, titanium dioxide and one or more iron oxides or several pigments and optionally one or more colourings, particularly 1.00 to 5.00 of a film coating comprising HPMC, MHPC, PEG, one or more silicates, titanium dioxide and one or more iron oxides.

In order to prepare the preparation according to the invention, the active substance is dissolved in a solvent, optionally in the presence of a moisture binder, sprayed onto the carriers, particularly finely divided, anhydrous lactose, lactose monohydrate, and microcrystalline cellulose as binders, mixed, screened, and then dried. The product obtained is optionally mixed with other carrier material, particularly microcrystalline cellulose and/or lactose, with breakdown agents, particularly cross-linked CMC Na, and finally with the flow agent, particularly magnesium stearate. The mixture thus obtained is then compressed in a suitable tablet press to produce the tablets according to the invention.

The compression forces needed to produce tablets of the required breaking strength and hence with the desired breakdown times are dependent on the shapes and sizes of the punching tools used. Preferably the compression force is in the range from 2-30 kN, particularly from 5-26 kN. Higher compression forces may result in tablets with a slower release of active substance. Lower compression forces may result in mechanically unstable tablets. The tablet cores may take various forms: round, doubly convex, and oval or oblong shapes are preferred.

Then a solution of the film-forming agent and plasticisers in water is prepared, the parting compounds and pigments which are insoluble therein are dispersed, and the resulting suspension is applied to the tablets.

The Examples that follow serve to illustrate the formulations according to the invention. They are intended solely as possible procedures described by way of example without restricting the invention to their contents.

### Example 1

Film-coated tablets are prepared consisting of:

### I. Composition

| | | | | volatile |
|---|---|---|---|---|
| | | mg/ | mg/film | constituent |
| | Ingredients | tablet | coating | mg/total |
| (01) | formula (IA) citrate | 1.585 | | |
| (02) | fine lactose | 79.415 | | |
| (03) | lactose monohydrate ( | 78.000 | | |
| (04) | microcryst. cellulose type 101 | 72.000 | | |
| (05) | hydroxypropylcellulose (Klucel EF | 2.400 | | |
| | Pharm) | | | |
| (06) | carboxymethylcell-NA (Ac-di-Sol) | 4.800 | | |
| (07) | vegetable magnesium stearate | 1.800 | | |
| (08) | Hypromellose (Methocel E5 | | 2.500 | |
| | Premium) | | | |
| (09) | Macrogol 6000 | | 0.250 | |
| (10) | titanium dioxide | | 1.250 | |
| (11) | talc | | 0.750 | |
| (12) | iron oxide yellow 17015 | | 0.125 | |
| (13) | iron oxide red 17009 | | 0.125 | |
| (14) | ethanol 96% | 26.880 | | 26.880 |
| (15) | purified water | 17.920 | 34.000 | 51.920 |
| | | 240.000 | 5.000 | 78.800 |

### II. Description of Product

| | tablets | film-coated tablet |
|---|---|---|
| Form | round, | round, |
| | convex (RC 13.5 mm), | convex (RC 13.5 mm), |
| | with facet | with facet |
| colour | white | salmon pink |
| nominal weight | 240 mg | 245 mg |
| diameter | approx. 9.0 mm | approx. 9.0 mm |
| height | approx. 3.5 mm | approx. 3.6 mm |
| breaking | approx. 75 N | approx. 100 N |
| strength | | |
| breakdown time | values measured: <5 min | values measured: <5 min |

### III. Preparation

### A) Tablets

1 batch of final mixture and tablets: 15000 g corresponds to 62500 tablets

1. Granulating Liquid

| | Place | |
|---|---|---|
| (15) | purified water and | 1120.000 g |
| (14) | ethanol 96% PAR INT | 1680.000 g |
| | in a suitable vessel (ambient temperature). | |
| | Then stir in, in succession, | |
| (05) | hydroxypropylcellulose (Klucel EF Pharm) INT | 150.000 g |
| | and | |
| (01) | formula (IA) citrate | 99.063 g |
| | and dissolve therein. | |
| | Solid content: 249.063 g | |
| | | 3049.063 g |

Process data:
Stirrer: SPN-Stirrer
speed/duration: approx. 250-450 rpm

2. Granules

| | Place | |
|---|---|---|
| (02) | fine lactose INT | 4963.437 g |
| (03) | lactose monohydrate (Tablettose) INT | 4875.000 g |
| | and | |
| (04) | microcryst. cellulose type 101 INT | 4500.000 g |
| | in a suitable one-pot granulator, | |
| | mix homogeneously and moisten with the | |
| | granulating liquid 1. | 3049.063 g |
| | Solid content: 249.063 g | |
| | granulate and then dry. | |
| | | 14587.500 g |

Process data:
Intensive mixer: Zanchetta Roto P 50

| | | | | temperature | final |
|---|---|---|---|---|---|
| | | mixing | | heating | product |
| | | speed | blender | jacket | temperature |
| | | (rpm) | (rpm) | (° C.) | (° C.) |
| operating | duration | 250 | — | RT | — |
| step | (min) | | | | |
| premixing | 3 | 250 | — | RT | — |
| moistening | approx. 5 | 250-300 | — | RT | — |
| rinsing | approx. 1 | 300 | — | RT | — |
| damp mixing | 2 | 250 | 1000 | RT | — |
| drying | approx.50 | 5 | — | to approx. | approx. 48 |
| | | | | 80 | |
| cooling | 15 | 5 | — | to approx. | <40 |
| | | | | 25 | |

nozzle head: 1.1 mm
spray pressure: approx. 2 bar
tilting angle: 100° (during drying and cooling)
   During the drying and cooling the mixer should operate intermittently, i.e. 1 minute mixing, then 2 minutes' rest.

3. Dry Screening
Comminute the dried granules using a suitable
screening machine.
Process data:
screening machine: Comil 197 S
screening size: RS 2007
spacer ring: DR 125

4. Final Mixture

| | | |
|---|---|---|
| | In a suitable gravity mixer mix the | 14587.500 g |
| | dry screened material 3. | |
| | with | |
| (07) | carboxymethylcell-NA, cross-linked (Ac-di-Sol) | 300.000 g |
| | INT | |
| | Then add | |
| (06) | vegetable magnesium stearate INT | 112.500 g |
| | prescreened to 0.5 mm and mix homogeneously. | |
| | | 15000.000 g |

Process data:
gravity mixer: Servolift Kubus 60 l
mixing speed: 10 rpm
number of
revolutions: 100 U (Ac-di-Sol INT)
   30 U (MgSt.INT)

5. Tablets

| | |
|---|---|
| In a suitable tablet press, | 15000.000 g |
| compress the | |
| final mixture 4. | |
| to form tablets. | |
| nominal weight: 240 mg | |

Process data:
tablet press: Korsch EKO
tool: 9 mm RC 13.5, doubly convex with facet+
BI logo
pressing speed stage 4
pressing force: approx. 11-12 kN

### B) Film-Coated Tablets

1 batch of 2640 g=11000 tablets
2695 g=11000 film-coated tablets

6. Coating Suspension/Solution

| | | | |
|---|---|---|---|
| (15) | purified water | 261.800 | g |
| (08) | Hypromellose (Methocel E5 Prem) INT | 27.500 | g |
| (07) | Macrogol 6000 INT | 2.750 | g |
| | Place (15) in a suitable container, stir in (08) and | | |
| | (07) at ambient temperature and dissolve (min. 15 | | |
| | minutes). | | |
| | Solid content 30.250 g | | |
| | | 292.050 | g |

7. Coating Suspension/Dispersion

| | | | |
|---|---|---|---|
| (15) | purified water | 112.200 | g |
| (10) | titanium dioxide INT | 13.750 | g |
| (11) | talc INT | 8.250 | g |
| (12) | iron oxide yellow 17015 INT | 1.375 | g |
| (13) | iron oxide red 17009 INT | 1.375 | g |
| | Place (15) in a suitable container, at ambient | | |
| | temperature suspend (10), (11), (12) and (13) | | |
| | therein using an Ultra-Turrax and stir for 30 | | |
| | minutes. | | |
| | Solid content 24.750 g | | |
| | | 136.950 | g |

8. Coating Suspension

| | | |
|---|---|---|
| coating suspension/solution | 6. | 292.050 g |
| coating suspension/dispersion | 7. | 136.950 g |
| Stir dispersion 7. into solution 6. and then stir | | |
| for 5 minutes. | | |
| Solid content 55.000 g | | |
| | | 429.000 g |

9. Film-Coating

| In a suitable film-coating apparatus | | |
|---|---|---|
| coat tablet cores | 5. | 2640.000 g |
| with coating suspension | 8. | 429.000 g |
| to a weight of 245 mg. | | |
| Solid content 55.000 g | | |
| | | 2695.000 g |

### Example 2

Corresponding non-coated tablets are prepared analogously to Example 1 by applying to the carrier material a solution of the active substance of formula (IA) in the form of the citrate dissolved in water and ethanol, but without the addition of hydroxypropylcellulose.

### Example 3

### I. Composition

| | | | | volatile |
|---|---|---|---|---|
| | | mg/ | mg/film | constituent |
| | constituents | tablet | coating | mg/total |
| (01) | formula (IA) citrate | 0.098 | | |
| (02) | fine lactose | 30.427 | | |
| (03) | lactose monohydrate | 29.000 | | |
| (04) | hydroxypropylcellulose (Klucel EF | 0.900 | | |
| | Pharm) | | | |
| (05) | microcryst. cellulose type 101 | 27.000 | | |
| (06) | carboxymethylcell-NA (Ac-di-Sol) | 1.800 | | |
| (07) | vegetable magnesium stearate | 0.675 | | |
| (08) | Hypromellose (Methocel E5 | | 1.500 | |
| | Premium) | | | |
| (09) | Macrogol 6000 | | 0.025 | |
| (10) | titanium dioxide | | 0.624 | |
| (11) | talc | | 0.375 | |
| (12) | iron oxide yellow 17015 | | 0.063 | |
| (13) | iron oxide red 17009 | | 0.063 | |
| (14) | ethanol 96% | 4.667 | | 4.667 |
| (15) | purified water | 3.020 | 18.709 | 21.829 |
| | | 90.000 | 2.500 | 26.496 |

### II. Description of Product

| | tablets | film-coated tablet |
|---|---|---|
| shape | round, convex | round, convex (RC 9 mm), |
| | (RC 9 mm), with facet | with facet |
| colour | white | salmon pink |
| nominal weight | 90 mg | 92.5 mg |
| diameter | approx. 6.0 mm | approx. 6.1 mm |
| Height | approx. 2.9 mm | approx. 3.0 mm |
| breaking | approx. 45 N | approx. 60 N |
| strength | | |
| breakdown time | values measured: <5 min | values measured: <5 min |

### III. Preparation

### A) Tablets

1 batch of final mixture and tables: 18000 g corresponds to 200000 tablets

1. Granulating Liquid

| | Place | |
|---|---|---|
| (15) | purified water and | 664.092 g |
| (14) | ethanol 96% PAR INT | 993.422 g |
| | in a suitable vessel (ambient temperature). | |
| | Then successively stir in | |
| (04) | hydroxypropylcellulose (Klucel EF Pharm) INT | 180.000 g |
| | and | |
| (01) | formula (IA) citrate | 39.600 g |
| | and dissolve therein. | |
| | Solid content: 219.600 g | |
| | | 1877.014 g |

Process data:
Stirrer: SPN-Stirrer
speed/duration: approx. 250-450 rpm

2. Granules

| | Place | |
|---|---|---|
| (02) | fine lactose INT | 6085.400 g |
| (03) | lactose monohydrate (Tablettose) INT | 5800.000 g |
| | in a suitable one-pot granulator, mix | |
| | homogeneously and moisten with the | |
| | granulating liquid 1. | 1877.014 g |
| | Solid content: 219.600 g | |
| | granulate and then dry. | |
| | | 12105.000 g |

Process data:
Intensive mixer: Zanchetta Roto P 50

| | | | | temperature | |
|---|---|---|---|---|---|
| | | mixing | | heating | final product |
| | | speed | blender | jacket | temperature |
| | | (rpm) | (rpm) | (° C.) | (° C.) |
| operating | duration | 250 | — | RT | — |
| step | (min) | | | | |
| premixing | 3 | 200-250 | — | RT | — |
| moistening | approx. 5 | 200-250 | — | RT | — |
| rinsing | approx. 1 | 200-250 | — | RT | — |
| damp mixing | 1 | 250 | 1000 | RT | — |
| drying | approx.50 | 5 | — | to approx. | approx. 48 |
| | | | | 80 | |
| cooling | 15 | 5 | — | to approx. | <40 |
| | | | | 25 | |

nozzle head: 1.1 mm

spray pressure: approx. 2 bar

tilting angle: 100° (during drying and cooling)

During the drying and cooling the mixer should operate continuously, 5 rpm.

3. Dry Screening
Comminute the dried granules using a suitable
screening machine.
Process data:
screening machine: Comil 197 S
screening size: RS 2007
spacer ring: DR 125

4. Final Mixture

| | | | |
|---|---|---|---|
| | In a suitable gravity mixer mix the | 12105.000 | g |
| | dry screened material 3. | | |
| | with | | |
| (05) | microcryst. cellulose type 101 INT | 5400.000 | g |
| (07) | carboxymethylcell-NA, cross-linked | 360.000 | g |
| | (Ac-di-Sol) INT Then add | | |
| (06) | vegetable magnesium stearate INT | 135.000 | g |
| | prescreened to 0.5 mm and mix | | |
| | homogeneously. | | |
| | | 18000.000 | g |

Process data:
gravity mixer: Servolift Kubus 60 I
mixing speed: 10 rpm
number of
revolutions: 100 U (Ac-di-Sol INT,
MCC type 101) 30 U (MgSt.INT)

5. Tablets

| | |
|---|---|
| In a suitable tablet press compress the | 18000.000 g |
| final mixture 4. | |
| to form tablets. | |
| nominal weight: 90 mg | |

Process data:
tablet press: Fette P1200
tool: 6 mm RC 9, biconvex
   with facet+BI logo
pressing speed 150.000 Tbl/h
pressing force: approx. 7-9 kN

### B) Film-Coated Tablets

1 batch of 2640 g=29333 tablets
2713 g=29333 film-coated tablets

6. Coating Suspension/Solution

| | | | |
|---|---|---|---|
| (15) | purified water | 384.063 | g |
| (08) | Hypromellose (Methocel E5 Prem) INT | 36.666 | g |
| (07) | Macrogol 6000 INT | 3.667 | g |
| | Place (15) in a suitable container, stir in (08) and | | |
| | (07) at ambient temperature and dissolve (min. 15 | | |
| | minutes). | | |
| | Solid content 40.333 g | | |
| | | 424.496 | g |

7. Coating Suspension/Dispersion

| | | | |
|---|---|---|---|
| (15) | purified water | 164.623 | g |
| (10) | titanium dioxide INT | 18.304 | g |
| (11) | talc INT | 11.000 | g |
| (12) | iron oxide yellow 17015 INT | 1.848 | g |
| (13) | iron oxide red 17009 INT | 1.848 | g |
| | Place (15) in a suitable container, suspend (10), | | |
| | (11), (12) and (13) therein at ambient temperature | | |
| | using an Ultra-Turrax and stir for 30 minutes. | | |
| | Solid content 33.000 g | | |
| | | 197.623 | g |

8. Coating Suspension

| | | |
|---|---|---|
| Coating suspension/solution | 6. | 424.496 g |
| Coating suspension/Dispersion | 7. | 197.623 g |
| Stir dispersion 7. into solution 6. and then | | |
| stir for 5 minutes. | | |
| Solid content 73.333 g | | |
| | | 622.119 g |

9. Film-Coating

| In a suitable film-coating apparatus | | |
|---|---|---|
| coat tablet cores | 5. | 2639.970 g |
| with coating suspension | 8. | 622.119 g |
| to a weight of 92.5 mg. | | |
| Solid content 73.333 g | | |
| | | 2713.303 g |

### Example 4

Investigating the Rate of Dissolution

The tablets according to Examples 1 and 2 are in each case dissolved in 900 ml of a simulated gastric fluid, pH 1.2, or simulated intestinal flora, pH 6.8 (0.05 M phosphate buffer) at a stirring speed of 50 rpm or 75 rpm, respectively. The content of dissolved compound of formula (IA) is determined by HPLC.

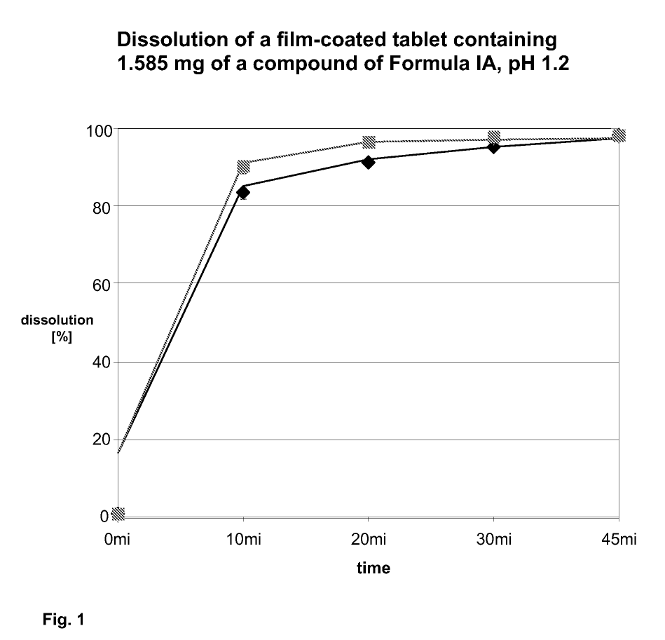

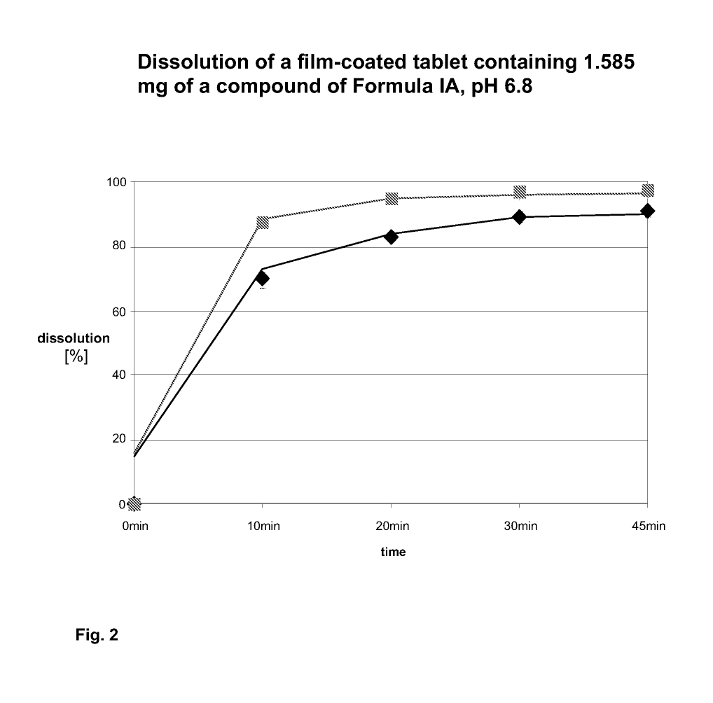

The progress of this dissolution over time is shown in FIGS. 1 and 2.

The symbols have the following meanings:

[symbol] Example 1 with moisture binders

[symbol] Example 2 without moisture binders

What is claimed is:

1. A pharmaceutical preparation comprising:
an active substance comprising one or more a monoamine neurotransmitter re-uptake inhibitors that have a 2,3-disubstituted tropane structure;
a solid carrier; and
a moisture binder.

2. The pharmaceutical preparation according to claim 1, wherein the active substance is a compound of formula I 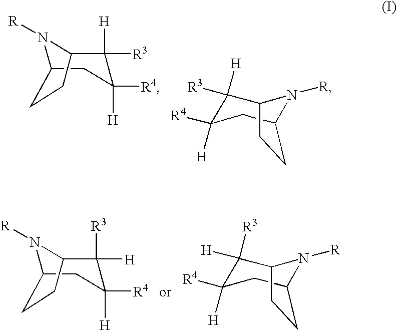
or a tautomer, a pharmaceutically acceptable salt, solvate, or a physiological functional derivative thereof, wherein
R is hydrogen, alkyl, alkenyl, alkynyl, cycloalkyl, cycloalkylalkyl or 2-hydroxyethyl;
R³ is CH₂—X—R′, where
X denotes O, S, or NR″; wherein R″ is hydrogen or alkyl; and
R′ is alkyl, alkenyl, alkynyl, cycloalkyl, cycloalkylalkyl, or —CO-alkyl;
heteroaryl, which may be mono- or polysubstituted by alkyl, cycloalkyl, or cycloalkylalkyl;
phenyl, which may be mono- or polysubstituted by a substituent selected from among halogen, CF₃, CN, alkoxy, alkyl, alkenyl, alkynyl, amino, nitro, and heteroaryl;
phenylphenyl;
pyridyl, which may be mono- or polysubstituted by a substituent selected from among halogen, CF₃, CN, alkoxy, alkyl, alkenyl, alkynyl, amino, nitro, and heteroaryl;
thienyl, which may be mono- or polysubstituted by a substituent selected from among halogen, CF₃, CN, alkoxy, alkyl, alkenyl, alkynyl, amino, nitro, and heteroaryl; or
benzyl, which may be mono- or polysubstituted by a substituent selected from among halogen, CF₃, CN, alkoxy, alkyl, alkenyl, alkynyl, amino, nitro, and heteroaryl; or
(CH₂)ₙCO₂R¹¹, COR¹¹, or CH₂R¹², wherein
R¹¹ is alkyl, cycloalkyl, or cycloalkylalkyl; phenyl, which may be mono- or polysubstituted by a substituent selected from among halogen, CF₃, CN, alkoxy, alkyl, alkenyl, alkynyl, amino, nitro, and heteroaryl; phenylphenyl; pyridyl which may be mono- or polysubstituted by a substituent selected from among halogen, CF₃, CN, alkoxy, alkyl, alkenyl, alkynyl, amino, nitro, and heteroaryl; or thienyl which may be mono- or polysubstituted by a substituent selected from among halogen, CF₃, CN, alkoxy, alkyl, alkenyl, alkynyl, amino, nitro, and heteroaryl; or benzyl;
n is 0 or 1; and
R¹² is O-phenyl, which may be mono- or polysubstituted by a substituent selected from among halogen, CF₃, CN, alkoxy, alkyl, alkenyl, alkynyl, amino, nitro, and heteroaryl; or
O—CO-phenyl, which may be mono- or polysubstituted by a substituent selected from among halogen, CF₃, CN, alkoxy, alkyl, alkenyl, alkynyl, amino, nitro, and heteroaryl; or
CH═NOR′; wherein R′ is hydrogen; alkyl, cycloalkyl, cycloalkylalkyl, alkenyl, alkynyl, or aryl, which may in turn be substituted by —COON, —COO-alkyl, —COO-cycloalkyl, or phenyl, which may be mono- or polysubstituted by a substituent selected from among halogen, CF₃, CN, alkoxy, alkyl, alkenyl, alkynyl, amino, and nitro;
R⁴ is phenyl, 3,4-methylenedioxyphenyl, benzyl, naphthyl, or heteroaryl, which may be mono- or polysubstituted by a substituent selected from among halogen, CF₃, CN, alkoxy, alkyl, alkenyl, alkynyl, amino, nitro, and heteroaryl.

3. The pharmaceutical preparation according to claim 1, wherein the active substance is a compound of formula I1: 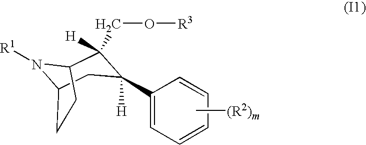
wherein
R¹ denotes a hydrogen atom or a C₁₋₆ alkyl group;
R² denotes a halogen atom or a CF₃ or cyano group;
R³ denotes a hydrogen atom or a C₁₋₆ alkyl group or C₃₋₆-cycloalkyl-C₁₋₃-alkyl group; and
m is 0 or an integer from 1 to 3;
or a tautomer, a pharmaceutically acceptable salt, solvate, or a physiologically functional derivative thereof.

4. The pharmaceutical preparation according to claim 1, wherein the active substance is selected form the group consisting of:
(1R,2R,3S)-2-(3-cyclopropyl-1,2,4-oxadiazol-5-yl)-3-(4-fluorophenyl)tropane;
(1R,2R,3S)-2-(3-phenyl-1,2,4-oxadiazol-5-yl)-3-(4-fluorophenyl)tropane;
(1R,2R,3S)-2-(3-phenyl-1,2,4-oxadiazol-5-yl)-3-(4-methylphenyl)-tropane;
(1R,2R,3S)-2-(3-benzyl-1,2,4-oxadiazol-5-yl)-3-(4-fluorophenyl)tropane;
(1R,2R,3S)-2-(3-(4-phenyl-phenyl)-1,2,4-oxadiazol-5-yl)-3-(4-fluorophenyl)tropane;
(1R,2R,3S)-2-(3-phenyl-1,2,4-oxadiazol-5-yl)-3-(2-naphthyl)tropane;
(1R,2R,3S)-3-(3,4-dichlorophenyl)tropane-2-aldoxime;
(1R,2R,3S)-3-(3,4-dichlorophenyl)-tropane-2-O-methyl-aldoxime;
(1R,2R,3S)-3-(3,4-dichlorophenyl)tropane-2-O-benzyl-aldoxime;
(1R,2R,3S)-3-(3,4-dichlorophenyl)tropane-2-O-ethoxycarbonylmethyl-aldoxime;
(1R,2R,3S)-3-(3,4-dichlorophenyl)tropane-2-O-methoxycarbonylmethyl-aldoxime;
(1R,2R,3S)-3-(3,4-dichlorophenyl)tropane-2-O-(1-ethoxycarbonyl-1,1-dimethyl-methyl)-aldoxime;
(1R,2R,3S)-3-(3,4-dichlorophenyl)tropane-2-O-carboxymethyl-2-aldoxime;
(1R,2R,3S)—N-normethyl-3-(3,4-dichlorophenyl)tropane-2-O-methyl-aldoxime;
(1R,2R,3S)—N-normethyl-3-(3,4-dichlorophenyl)tropane-2-O-benzyl-aldoxime;
(1R,2R,3S)-3-(4-methylphenyl)tropane-2-O-methyl-aldoxime;
(1R,2R,3S)-3-(3,4-dichlorophenyl)tropane-2-O-(1,1-dimethylethyl)-aldoxime;
(1R,2R,3S)-3-(4-chlorophenyl)tropane-2-O-aldoxime;
(1R,2R,3S)-3-(4-chlorophenyl)tropane-2-O-methylaldoxime hydrochloride;
(1R,2R,3S)-3-(4-chlorophenyl)tropane-2-O-methoxycarbonylmethyl-aldoxime;
(1R,2R,3S)-3-(3,4-dichlorophenyl)tropane-2-O— (2-propynyl)-aldoxime;
(1R,2R,3S)-3-(3,4-dichlorophenyl)tropane-2-O-(2-methylpropyl)-aldoxime;
(1R,2R,3S)-3-(3,4-dichlorophenyl)tropane-2-β-cyclopropylmethyl-aldoxime;
(1R,2R,3S)-3-(3,4-dichlorophenyl)tropane-2-O-ethyl-aldoxime;
(1R,2R,3S)-2-methoxymethyl-3-(3,4-dichlorophenyl)-tropane;
(1R,2R,3S)-2-isopropoxymethyl-3-(3,4-dichlorophenyl)-tropane;
(1R,2R,3S)-2-ethoxymethyl-3-(3,4-dichlorophenyl)-tropane;
(1R,2R,3S)-2-ethoxymethyl-3-(3,4-dichlorophenyl)-nortropane;
(1R,2R,3S)-2-cyclopropylmethyloxymethyl-3-(3,4-dichlorophenyl)-tropane;
(1R,2R,3S)-2-methoxymethyl-3-(4-chlorophenyl)-tropane;
(1R,2R,3S)—N-normethyl-2-methoxymethyl-3-(4-chlorophenyl)-tropane;
(1R,2R,3S)-2-ethoxymethyl-3-(4-chlorophenyl)-tropane;
(1R,2R,3S)—N-normethyl-2-methoxymethyl-3-(3,4-dichlorophenyl)-tropane;
(1R,2R,3S)—N-normethyl-2-ethoxymethyl-3-(3,4-dichlorophenyl)-tropane;
(1R,2R,3S)—N-normethyl-2-ethoxymethyl-3-(4-chlorophenyl)-tropane;
(1R,2R,3S)—N-normethyl-2-cyclopropylmethyloxymethyl-3-(4-chlorophenyl)-tropane;
(1R,2R,3S)-2-cyclopropylmethyloxymethyl-3-(4-chlorophenyl)-tropane;
(1R,2R,3S)-2-ethylthiomethyl-3-(3,4-dichlorophenyl)-tropane;
(1R,2R,3S)-2-hydroxymethyl-3-(4-fluorophenyl)tropane;
(1R,2R,3S)-2-hydroxymethyl-3-(3,4-dichlorophenyl)tropane;
(1R,2R,3S)—N-normethyl-N-(tert-butoxycarbonyl)-2-hydroxymethyl-3-(3,4-dichlorophenyl)tropane;
(1R,2R,3S)-2-hydroxymethyl-3-(4-chlorophenyl)tropane;
(1R,2R,3S)-2-(3-(2-furanyl)-1,2,4-oxadiazol-5-yl)-3-(3,4-dichlorophenyl)-tropane;
(1R,2R,3S)-2-(3-(3-pyridyl)-1,2,4-oxadiazol-5-yl)-3-(3,4-dichlorophenyl)-tropane;
(1R,2R,3S)—N-normethyl-N-allyl-2-(3-(4-pyridyl)-1,2,4-oxadiazol-5-yl)-3-(3,4-dichlorophenyl)-tropane;
(1R,2R,3S)—N-normethyl-N-ethyl-2-(3-(4-pyridyl)-1,2,4-oxadiazol-5-yl)-3-(3,4-dichlorophenyl)-tropane;
(1R,2R,3S)—N-normethyl-N-(2-hydroxyethyl)-2-(3-(4-pyridyl)-1,2,4-oxadiazol-5-yl)-3-(3,4-dichlorophenyl)-tropane;
(1R,2R,3S)—N-normethyl-2-(3-(4-pyridyl)-1,2,4-oxadiazol-5-yl)-3-(3,4-dichlorophenyl)-tropane;
(1R,2R,3S)—N-normethyl-N-allyl-2-(3-(3-pyridyl)-1,2,4-oxadiazol-5-yl)-3-(3,4-dichlorophenyl)-tropane;
(1R,2R,3S)—N-normethyl-N-allyl-2-(3-(2-pyridyl)-1,2,4-oxadiazol-5-yl)-3-(3,4-dichlorophenyl)-tropane;
(1R,2R,3S)-2-(3-(2-thienyl)-1,2,4-oxadiazol-5-yl)-3-(4-chlorophenyl)-tropane;
(1R,2R,3S)-2-(3-(2-thienyl)-1,2,4-oxadiazol-5-yl)-3-(3,4-dichlorophenyl)-tropane;
(1R,2R,3S)-2-(3-(4-pyridyl)-1,2,4-oxadiazol-5-yl)-3-(3,4-dichlorophenyl)-tropane;
(1R,2R,3S)-2-(3-(2-pyridyl)-1,2,4-oxadiazol-5-yl)-3-(3,4-dichlorophenyl)-tropane;
(1R,2R,3S)-2-(3-(4-pyridyl)-1,2,4-oxadiazol-5-yl)-3-(4-chlorophenyl)-tropane;
(1R,2R,3S)-2-(3-(3-pyridyl)-1,2,4-oxadiazol-5-yl)-3-(4-chlorophenyl)-tropane;
(1R,2R,3S)-2-(3-2-pyridyl)-1,2,4-oxadiazol-5-yl)-3-(4-chlorophenyl)-tropane;
(1R,2R,3S)-2-(3-phenyl-1,2,4-oxadiazol-5-yl)-3-(4-fluorophenyl)-tropane;
(1R,2R,3S)-2-(3-phenyl-1,2,4-oxadiazol-5-yl)-3-(4-methylphenyl)-tropane;
(1R,2R,3S)-2-(3-benzyl-1,2,4-oxadiazol-5-yl)-3-(4-fluorophenyl)-tropane;
(1R,2R,3S)-2-(3-(4-phenylphenyl)-1,2,4-oxadiazol-5-yl)-3-(4-fluorophenyl)-tropane;
(1R,2R,3S)-2-(3-phenyl-1,2,4-oxadiazol-5-yl)-3-(2-naphthyl)-tropane;
(1R,2R,3S)-2-(4-chlorophenoxy-methyl)-3-(4-fluorophenyl)-tropane;
(1R,2R,3S)-2-(4-chlorophenoxy-methyl)-3-(4-fluorophenyl)-tropane;
(1R,2R,3S)-2-(4-chlorophenoxy-methyl)-3-(3,4-dichlorophenyl)-tropane;
(1R,2R,3S)-2-(4-chlorophenoxy-methyl)-3-(4-methylphenyl)-tropane;
(1R,2R,3S)-2-(4-benzoyloxy-methyl)-3-(4-fluorophenyl)-tropane;
(1R,2R,3S)-2-carbomethoxy-3-(2-naphthyl)-tropane;
(1R,2R,3S)-2-carbomethoxy-3-(3,4-dichlorophenyl)-tropane;
(1R,2R,3S)-2-carbomethoxy-3-benzyl-tropane;
(1R,2R,3S)-2-carbomethoxy-3-(4-chlorophenyl)-tropane;
(1R,2R,3S)-2-carbomethoxy-3-(4-methylphenyl)-tropane;
(1R,2R,3S)-2-carbomethoxy-3-(1-naphthyl)-tropane;
(1R,2R,3S)-2-carbomethoxy-3-(4-phenylphenyl)-tropane;
(1R,2R,3S)-2-carbomethoxy-3-(4-t-butyl-phenyl)-tropane;
(1R,2R,3S)-2-(4-fluorobenzoyl)-3-(4-fluorophenyl)-tropane;
and tautomers, pharmaceutically acceptable salts, solvates, and physiological functional derivatives thereof, and mixtures thereof.

5. The pharmaceutical preparation according to claim 1, wherein the active substance is a compound of formula IA 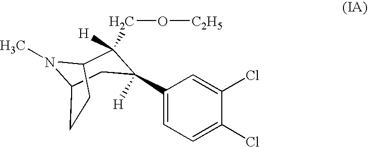
or a tautomer, a pharmaceutically acceptable salt, solvate, or a physiological functional derivative thereof.

6. The pharmaceutical preparation according to claim 1, wherein the solid carrier is selected from the group consisting of: carbohydrates, sugar alcohols, and dry binders, and combinations thereof.

7. The pharmaceutical preparation according to claim 1, wherein the moisture binder is selected from the group consisting of: polyvinylpyrrolidone (Povidone), copolymers of vinylpyrrolidone with other vinyl derivatives (Copovidone), cellulose derivatives, methylhydroxypropylcellulose, methylcellulose, and hydroxypropylcellulose, and combinations thereof.

8. The pharmaceutical preparation according to claim 1, further comprising an excipient selected from the group consisting of: moisture binders, lubricants, breakdown agents, parting compounds, and wetting agents, and combinations thereof.

9. The pharmaceutical preparation according to claim 1 in the form of a film-coated tablet.

10. The pharmaceutical preparation according to claim 1, wherein:
the active substance is present in an amount about 0.01 to 5.00 wt. %;
the solid carrier is present in an amount about 80.00 to 95.00 wt. %; and
the moisture binder is present in an amount about 1.00 to 10.00 wt. %.

11. A process for preparing a pharmaceutical preparation comprising: dissolving an active substance comprising one or more monoamine neurotransmitter re-uptake inhibitors that have a 2,3-disubstituted tropane structure in a suitable solvent in the presence of a moisture binder; and spraying the resulting solution onto a solid carrier to form a solid mixture.

12. The process according to claim 11, wherein the active substance is a compound of formula I 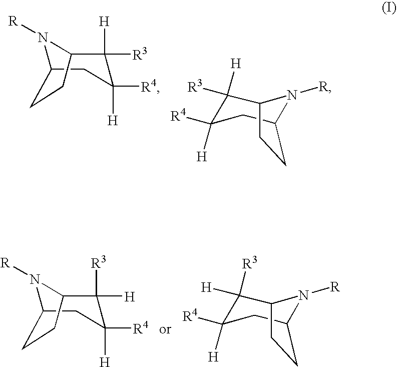
or a tautomer, a pharmaceutically acceptable salt, solvate, or a physiological functional derivative thereof, wherein
R is hydrogen, alkyl, alkenyl, alkynyl, cycloalkyl, cycloalkylalkyl or 2-hydroxyethyl;
R³ is CH₂—X—R′, where
X denotes O, S, or NR″; wherein R″ is hydrogen or alkyl; and
R′ is alkyl, alkenyl, alkynyl, cycloalkyl, cycloalkylalkyl, or —CO-alkyl;
heteroaryl, which may be mono- or polysubstituted by alkyl, cycloalkyl, or cycloalkylalkyl;
phenyl, which may be mono- or polysubstituted by a substituent selected from among halogen, CF₃, CN, alkoxy, alkyl, alkenyl, alkynyl, amino, nitro, and heteroaryl;
phenylphenyl;
pyridyl, which may be mono- or polysubstituted by a substituent selected from among halogen, CF₃, CN, alkoxy, alkyl, alkenyl, alkynyl, amino, nitro, and heteroaryl;
thienyl, which may be mono- or polysubstituted by a substituent selected from among halogen, CF₃, CN, alkoxy, alkyl, alkenyl, alkynyl, amino, nitro, and heteroaryl; or
benzyl, which may be mono- or polysubstituted by a substituent selected from among halogen, CF₃, CN, alkoxy, alkyl, alkenyl, alkynyl, amino, nitro, and heteroaryl; or
(CH₂)ₙCO₂R¹¹, COR¹¹, or CH₂R¹², wherein
R¹¹ is alkyl, cycloalkyl, or cycloalkylalkyl; phenyl, which may be mono- or polysubstituted by a substituent selected from among halogen, CF₃, CN, alkoxy, alkyl, alkenyl, alkynyl, amino, nitro, and heteroaryl; phenylphenyl; pyridyl which may be mono- or polysubstituted by a substituent selected from among halogen, CF₃, CN, alkoxy, alkyl, alkenyl, alkynyl, amino, nitro, and heteroaryl; or thienyl which may be mono- or polysubstituted by a substituent selected from among halogen, CF₃, CN, alkoxy, alkyl, alkenyl, alkynyl, amino, nitro, and heteroaryl; or benzyl;
n is 0 or 1; and
R¹² is O-phenyl, which may be mono- or polysubstituted by a substituent selected from among halogen, CF₃, CN, alkoxy, alkyl, alkenyl, alkynyl, amino, nitro, and heteroaryl; or
O—CO-phenyl, which may be mono- or polysubstituted by a substituent selected from among halogen, CF₃, CN, alkoxy, alkyl, alkenyl, alkynyl, amino, nitro, and heteroaryl; or
CH═NOR′; wherein R′ is hydrogen; alkyl, cycloalkyl, cycloalkylalkyl, alkenyl, alkynyl, or aryl, which may in turn be substituted by —COON, —COO-alkyl, —COO-cycloalkyl, or phenyl, which may be mono- or polysubstituted by a substituent selected from among halogen, CF₃, CN, alkoxy, alkyl, alkenyl, alkynyl, amino, and nitro;
R⁴ is phenyl, 3,4-methylenedioxyphenyl, benzyl, naphthyl, or heteroaryl, which may be mono- or polysubstituted by a substituent selected from among halogen, CF₃, CN, alkoxy, alkyl, alkenyl, alkynyl, amino, nitro, and heteroaryl.

13. The process according to claim 11, wherein the active substance is a compound of formula I1: 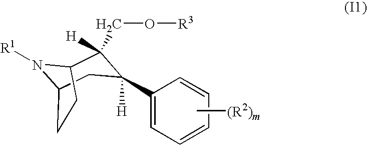
wherein
R¹ denotes a hydrogen atom or a C₁₋₆ alkyl group;
R² denotes a halogen atom or a CF₃ or cyano group;
R³ denotes a hydrogen atom or a C₁₋₆ alkyl group or C₃₋₆-cycloalkyl-C₁₋₃-alkyl group; and
m is 0 or an integer from 1 to 3;
or a tautomer, a pharmaceutically acceptable salt, solvate, or a physiologically functional derivative thereof.

14. The process according to claim 11, wherein the active substance is selected form the group consisting of:
(1R,2R,3S)-2-(3-cyclopropyl-1,2,4-oxadiazol-5-yl)-3-(4-fluorophenyl)tropane;
(1R,2R,3S)-2-(3-phenyl-1,2,4-oxadiazol-5-yl)-3-(4-fluorophenyl)tropane;
(1R,2R,3S)-2-(3-phenyl-1,2,4-oxadiazol-5-yl)-3-(4-methylphenyl)-tropane;
(1R,2R,3S)-2-(3-benzyl-1,2,4-oxadiazol-5-yl)-3-(4-fluorophenyl)tropane;
(1R,2R,3S)-2-(3-(4-phenyl-phenyl)-1,2,4-oxadiazol-5-yl)-3-(4-fluorophenyl)tropane;
(1R,2R,3S)-2-(3-phenyl-1,2,4-oxadiazol-5-yl)-3-(2-naphthyl)tropane;
(1R,2R,3S)-3-(3,4-dichlorophenyl)tropane-2-aldoxime;
(1R,2R,3S)-3-(3,4-dichlorophenyl)-tropane-2-O-methyl-aldoxime;
(1R,2R,3S)-3-(3,4-dichlorophenyl)tropane-2-O-benzyl-aldoxime;
(1R,2R,3S)-3-(3,4-dichlorophenyl)tropane-2-O-ethoxycarbonylmethyl-aldoxime;
(1R,2R,3S)-3-(3,4-dichlorophenyl)tropane-2-O-methoxycarbonylmethyl-aldoxime;
(1R,2R,3S)-3-(3,4-dichlorophenyl)tropane-2-O-(1-ethoxycarbonyl-1,1-dimethyl-methyl)-aldoxime;
(1R,2R,3S)-3-(3,4-dichlorophenyl)tropane-2-O-carboxymethyl-2-aldoxime;
(1R,2R,3S)—N-normethyl-3-(3,4-dichlorophenyl)tropane-2-O-methyl-aldoxime;
(1R,2R,3S)—N-normethyl-3-(3,4-dichlorophenyl)tropane-2-O-benzyl-aldoxime;
(1R,2R,3S)-3-(4-methylphenyl)tropane-2-O-methyl-aldoxime;
(1R,2R,3S)-3-(3,4-dichlorophenyl)tropane-2-O-(1,1-dimethylethyl)-aldoxime;
(1R,2R,3S)-3-(4-chlorophenyl)tropane-2-O-aldoxime;
(1R,2R,3S)-3-(4-chlorophenyl)tropane-2-O-methylaldoxime hydrochloride;
(1R,2R,3S)-3-(4-chlorophenyl)tropane-2-O-methoxycarbonylmethyl-aldoxime;
(1R,2R,3S)-3-(3,4-dichlorophenyl)tropane-2-O— (2-propynyl)-aldoxime;
(1R,2R,3S)-3-(3,4-dichlorophenyl)tropane-2-O-(2-methylpropyl)-aldoxime;
(1R,2R,3S)-3-(3,4-dichlorophenyl)tropane-2-β-cyclopropylmethyl-aldoxime;
(1R,2R,3S)-3-(3,4-dichlorophenyl)tropane-2-O-ethyl-aldoxime;
(1R,2R,3S)-2-methoxymethyl-3-(3,4-dichlorophenyl)-tropane;
(1R,2R,3S)-2-isopropoxymethyl-3-(3,4-dichlorophenyl)-tropane;
(1R,2R,3S)-2-ethoxymethyl-3-(3,4-dichlorophenyl)-tropane;
(1R,2R,3S)-2-ethoxymethyl-3-(3,4-dichlorophenyl)-nortropane;
(1R,2R,3S)-2-cyclopropylmethyloxymethyl-3-(3,4-dichlorophenyl)-tropane;
(1R,2R,3S)-2-methoxymethyl-3-(4-chlorophenyl)-tropane;
(1R,2R,3S)—N-normethyl-2-methoxymethyl-3-(4-chlorophenyl)-tropane;
(1R,2R,3S)-2-ethoxymethyl-3-(4-chlorophenyl)-tropane;
(1R,2R,3S)—N-normethyl-2-methoxymethyl-3-(3,4-dichlorophenyl)-tropane;
(1R,2R,3S)—N-normethyl-2-ethoxymethyl-3-(3,4-dichlorophenyl)-tropane;
(1R,2R,3S)—N-normethyl-2-ethoxymethyl-3-(4-chlorophenyl)-tropane;
(1R,2R,3S)—N-normethyl-2-cyclopropylmethyloxymethyl-3-(4-chlorophenyl)-tropane;
(1R,2R,3S)-2-cyclopropylmethyloxymethyl-3-(4-chlorophenyl)-tropane;
(1R,2R,3S)-2-ethylthiomethyl-3-(3,4-dichlorophenyl)-tropane;
(1R,2R,3S)-2-hydroxymethyl-3-(4-fluorophenyl)tropane;
(1R,2R,3S)-2-hydroxymethyl-3-(3,4-dichlorophenyl)tropane;
(1R,2R,3S)—N-normethyl-N-(tert-butoxycarbonyl)-2-hydroxymethyl-3-(3,4-dichlorophenyl)tropane;
(1R,2R,3S)-2-hydroxymethyl-3-(4-chlorophenyl)tropane;
(1R,2R,3S)-2-(3-(2-furanyl)-1,2,4-oxadiazol-5-yl)-3-(3,4-dichlorophenyl)-tropane;
(1R,2R,3S)-2-(3-(3-pyridyl)-1,2,4-oxadiazol-5-yl)-3-(3,4-dichlorophenyl)-tropane;
(1R,2R,3S)—N-normethyl-N-allyl-2-(3-(4-pyridyl)-1,2,4-oxadiazol-5-yl)-3-(3,4-dichlorophenyl)-tropane;
(1R,2R,3S)—N-normethyl-N-ethyl-2-(3-(4-pyridyl)-1,2,4-oxadiazol-5-yl)-3-(3,4-dichlorophenyl)-tropane;
(1R,2R,3S)—N-normethyl-N-(2-hydroxyethyl)-2-(3-(4-pyridyl)-1,2,4-oxadiazol-5-yl)-3-(3,4-dichlorophenyl)-tropane;
(1R,2R,3S)—N-normethyl-2-(3-(4-pyridyl)-1,2,4-oxadiazol-5-yl)-3-(3,4-dichlorophenyl)-tropane;
(1R,2R,3S)—N-normethyl-N-allyl-2-(3-(3-pyridyl)-1,2,4-oxadiazol-5-yl)-3-(3,4-dichlorophenyl)-tropane;
(1R,2R,3S)—N-normethyl-N-allyl-2-(3-(2-pyridyl)-1,2,4-oxadiazol-5-yl)-3-(3,4-dichlorophenyl)-tropane;
(1R,2R,3S)-2-(3-(2-thienyl)-1,2,4-oxadiazol-5-yl)-3-(4-chlorophenyl)-tropane;
(1R,2R,3S)-2-(3-(2-thienyl)-1,2,4-oxadiazol-5-yl)-3-(3,4-dichlorophenyl)-tropane;
(1R,2R,3S)-2-(3-(4-pyridyl)-1,2,4-oxadiazol-5-yl)-3-(3,4-dichlorophenyl)-tropane;
(1R,2R,3S)-2-(3-(2-pyridyl)-1,2,4-oxadiazol-5-yl)-3-(3,4-dichlorophenyl)-tropane;
(1R,2R,3S)-2-(3-(4-pyridyl)-1,2,4-oxadiazol-5-yl)-3-(4-chlorophenyl)-tropane;
(1R,2R,3S)-2-(3-(3-pyridyl)-1,2,4-oxadiazol-5-yl)-3-(4-chlorophenyl)-tropane;
(1R,2R,3S)-2-(3-2-pyridyl)-1,2,4-oxadiazol-5-yl)-3-(4-chlorophenyl)-tropane;
(1R,2R,3S)-2-(3-phenyl-1,2,4-oxadiazol-5-yl)-3-(4-fluorophenyl)-tropane;
(1R,2R,3S)-2-(3-phenyl-1,2,4-oxadiazol-5-yl)-3-(4-methylphenyl)-tropane;
(1R,2R,3S)-2-(3-benzyl-1,2,4-oxadiazol-5-yl)-3-(4-fluorophenyl)-tropane;
(1R,2R,3S)-2-(3-(4-phenylphenyl)-1,2,4-oxadiazol-5-yl)-3-(4-fluorophenyl)-tropane;
(1R,2R,3S)-2-(3-phenyl-1,2,4-oxadiazol-5-yl)-3-(2-naphthyl)-tropane;
(1R,2R,3S)-2-(4-chlorophenoxy-methyl)-3-(4-fluorophenyl)-tropane;
(1R,2R,3S)-2-(4-chlorophenoxy-methyl)-3-(4-fluorophenyl)-tropane;
(1R,2R,3S)-2-(4-chlorophenoxy-methyl)-3-(3,4-dichlorophenyl)-tropane;
(1R,2R,3S)-2-(4-chlorophenoxy-methyl)-3-(4-methylphenyl)-tropane;
(1R,2R,3S)-2-(4-benzoyloxy-methyl)-3-(4-fluorophenyl)-tropane;
(1R,2R,3S)-2-carbomethoxy-3-(2-naphthyl)-tropane;
(1R,2R,3S)-2-carbomethoxy-3-(3,4-dichlorophenyl)-tropane;
(1R,2R,3S)-2-carbomethoxy-3-benzyl-tropane;
(1R,2R,3S)-2-carbomethoxy-3-(4-chlorophenyl)-tropane;
(1R,2R,3S)-2-carbomethoxy-3-(4-methylphenyl)-tropane;
(1R,2R,3S)-2-carbomethoxy-3-(1-naphthyl)-tropane;
(1R,2R,3S)-2-carbomethoxy-3-(4-phenylphenyl)-tropane;
(1R,2R,3S)-2-carbomethoxy-3-(4-t-butyl-phenyl)-tropane;
(1R,2R,3S)-2-(4-fluorobenzoyl)-3-(4-fluorophenyl)-tropane;
and tautomers, pharmaceutically acceptable salts, solvates, and physiological functional derivatives thereof, and mixtures thereof.

15. The process according to claim 11, wherein the active substance is a compound of formula IA 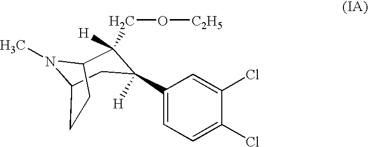
or a tautomer, a pharmaceutically acceptable salt, solvate, or a physiological functional derivative thereof.

16. The process according to claim 11, wherein the solid carrier is selected from the group consisting of: carbohydrates, sugar alcohols, and dry binders, and combinations thereof.

17. The process according to claim 11, wherein the moisture binder is selected from the group consisting of: polyvinylpyrrolidone (Povidone), copolymers of vinylpyrrolidone with other vinyl derivatives (Copovidone), cellulose derivatives, methylhydroxypropylcellulose, methylcellulose, and hydroxypropylcellulose, and combinations thereof.

18. The process according to claim 11, further comprising adding an excipient to the solid mixture, the excipient selected from the group consisting of: moisture binders, lubricants, breakdown agents, parting compounds, and wetting agents, and combinations thereof.

19. The process according to claim 11, wherein the solid mixture is formed into a film-coated tablet.

20. The pharmaceutical preparation according to claim 1, wherein:
the active substance is present in an amount about 0.01 to 5.00 wt. %;
the solid carrier is present in an amount about 80.00 to 95.00 wt. %; and
the moisture binder is present in an amount about 1.00 to 10.00 wt. %.
